(19) United States
(12) Patent Application Publication
(10) Pub. No.: US 2015/0154757 A1
(43) Pub. Date: Jun. 4, 2015

(54) IMAGE PROCESSOR, TREATMENT SYSTEM, AND IMAGE PROCESSING METHOD

(71) Applicant: KABUSHIKI KAISHA TOSHIBA, Tokyo (JP)

(72) Inventors: Yukinobu SAKATA, Kawasaki (JP); Yasunori Taguchi, Kawasaki (JP); Ryusuke Hirai, Tokyo (JP); Kyoka Sugiura, Kawasaki (JP); Tomoyuki Takeguchi, Kawasaki (JP); Takeshi Mita, Yokohama (JP); Shinya Fukushima, Tokyo (JP)

(21) Appl. No.: 14/476,118

(22) Filed: Sep. 3, 2014

(30) Foreign Application Priority Data
Nov. 29, 2013 (JP) 2013-247287

(51) Int. Cl. G06T 7/00; A61N 5/10; A61B 6/00; G06T 11/60

(52) U.S. Cl. CPC G06T 7/0026; G06T 11/60; A61N 5/1077; A61N 5/103; A61B 6/48; G06T 2207/10116; G06T 2207/30004

(57) ABSTRACT

According to an image processor of one embodiment, a first acquirer acquires a first perspective image of a target viewed in a first direction. A second acquirer acquires a second perspective image of the target viewed in a second direction at a time different from when the first acquirer acquires the first perspective image. The second direction is substantially the same as the first direction. The second time is different from the first time. A point acquirer acquires first information indicating a first position of a first point on the first perspective image, and a second information indicating a second position of a second point on the second perspective image. An image generator generates an image that is based on the first and second perspective images, wherein a first coordinate of the first perspective image is changed so that the first point corresponds to the second point.

### CROSS-REFERENCE TO RELATED APPLICATION

This application is based upon sod claims the benefit of priority from Japanese Patent Application No. 2013-247287, filed Nov. 29, 2013; the entire contents of which are incorporated herein by reference.

Embodiments described herein relate generally to an image processor, a treatment system, and an image processing method

### BACKGROUND

For radiotherapy treatments, schedules or plans are oracle based on previously-captured perspective images of a patient so that a locus of a disease in the body of the patient is precisely irradiated. An irradiation direction, an irradiation strength, and the like, are determined when a plan is made.

There are positioning apparatuses and methods for aligning a position, at the planning time, of a radiographic referential image of an object to be irradiated, to a position of the target at the time of actual irradiation. There is technology of displaying a guideline based on the epipolar geometry when a positioning process is performed. However, a guideline using an epipolar line is difficult to be displayed on images having projection planes which do not intersect each other.

### BRIEF DESCRIPTION Of THE DRAWINGS

FIG. 1 is a block diagram illustrating an example of treatment system according to embodiments.

FIG. 2 is a diagram illustrating a positional relationship of the radiographic imaging apparatus.

FIG. 3 is a diagram illustrating an example of a displayed image in eluding a first image, a second image, and a differential image as a similarity image with lower similarity, which are arranged in a line.

FIG. 4 is a diagram illustrating an example of a displayed image including a first image, a second image, and a differential image as a similarity image with higher similarity, which are arranged in a line.

FIG. 5 is a diagram illustrating a state of a differential image as a similarity image, on which an image representing a position of a point is superimposed.

FIG. 6 is a diagram illustrating a displayed image in which a superimposed image as a similarity image is superimposed on the second image.

FIG. 7 is a diagram illustrating a displayed image including the first image, the second image, and a similarity image based on an image rotated, increased or decreased in size, which are arranged in a line;

FIG. 8 is a diagram illustrating a displayed image including the first image, the second image, and an SSD map as a similarity image, which are arranged in a line;

FIG. 9 is a diagram illustrating a displayed image including the first image, the second image, and a patched image as a similarity image, which are arranged in a line.

FIG. 10 is a flowchart illustrating processes performed by the image processor according to one or more embodiments.

### DETAILED DESCRIPTION

According to one embodiment, an image processor may include, but is not limited to, a first acquirer, a second acquirer, a point acquirer, and an image generator. The first acquirer acquires, a first perspective image of a target viewed in a first direction at a first time. The second acquirer acquires a second perspective image of the target viewed in a second direction at a second time. The second direction is substantially the same as the first direction. The second time is different from the first time. The point acquirer acquires first information indicating a first position of a first point designated on the first perspective image, and second information indicating a second position of a second point designated on the second perspective image. The image generator generates a first image that is based on the first and second perspective images, wherein a first coordinate of the first perspective image is changed, so that the first point corresponds to the second point.

According to another embodiment, a treatment system may include, hut is not limited to, an linage processor, a radiographic imaging apparatus, a display system, an operation device, and a treatment apparatus. The image processor may include, but is not limited to, a first acquirer, a second acquirer, a point acquirer, and an image generator. The first acquirer acquires a first perspective image of a target viewed in a first direction at a first time. The second acquirer acquires a second perspective image of the target viewed in a second direction at a second time. The second direction is substantially the same as the first direction. The second time is different from the first time. The point acquirer acquires first information indicating a first, position of a first point designated on the first perspective image, and second information indicating a second position of a second point designated on the second perspective image. The image generator generates a first image that is based on the first and second perspective images, wherein a first coordinate of the first perspective image is changed so that the first point corresponds to the second point. The radiographic imaging apparatus captures the first or second perspective image of the target. The display system, displays the first and second perspective images. The operation device is used to input the first and second information to the point acquirer. The treatment apparatus is used to perform a treatment on the target based on the first image.

According to still another embodiment an image processing method may include, but is not limited to, the following processes. A first perspective image of a target viewed in a first direction at a first time is acquired. A second perspective image of the target viewed in a second direction at a second time is acquired. The second direction is substantially the same as the first direction. The second time is different from the first time. First Information indicating a first position of a first point designated on the first perspective image is acquired. Second information indicating a second position of a second point designated on the second perspective image is acquired A first image is generated. The first image is based on the first and second perspective images, wherein a first coordinate of the first perspective image is changed so that the first point corresponds to the second point.

In some embodiments, the point acquirer acquires at least one of an updated one of the first information indicating the first position updated, and an updated one of the second information indicating the second position updated. The image generator updates the first image based on the updated ones of the first and second informations.

In some embodiments, the image generator generates a second image that is based on an image in a first region of the first perspective image and based on an image in a second region of the second perspective image. The image in the first region includes the first point. The image in the second region includes the second point.

In some embodiments, the image generator superimposes, on the second image, any one of a first mark, specifying the first position and a second mark specifying the second position.

In some embodiments, the image generator generates any one of: a differential image between the first and second perspective images; a superimposed image of the differential image over the first image; and an image having a plurality of sectioned regions, each of which is a corresponding part of the first or second perspective image.

In some embodiments, the image generator generates any one of an image generated using a square sum of a difference in pixel value between the first and second perspective images; an image generated using an absolute sum of the difference in pixel value; and an image generated using a normalized mutual correlation between pixel values of the first and second perspective images.

In some embodiments, the image generator generates an image based on the first or second perspective image that is rotated or changed in size.

In some embodiments, the image generator generates a frame image representing the outline of the first image.

In some embodiments, the image generator changes a color of the frame image in accordance with a similarity between the first and second perspective images.

Various embodiments will be described hereinafter with reference to the accompanying drawings. The terms s“first” and “second” to be used in the descriptions made with reference to the illustrative drawings correspond respectively to “second” and “first” In the descriptions without referring to the drawings and the claims.

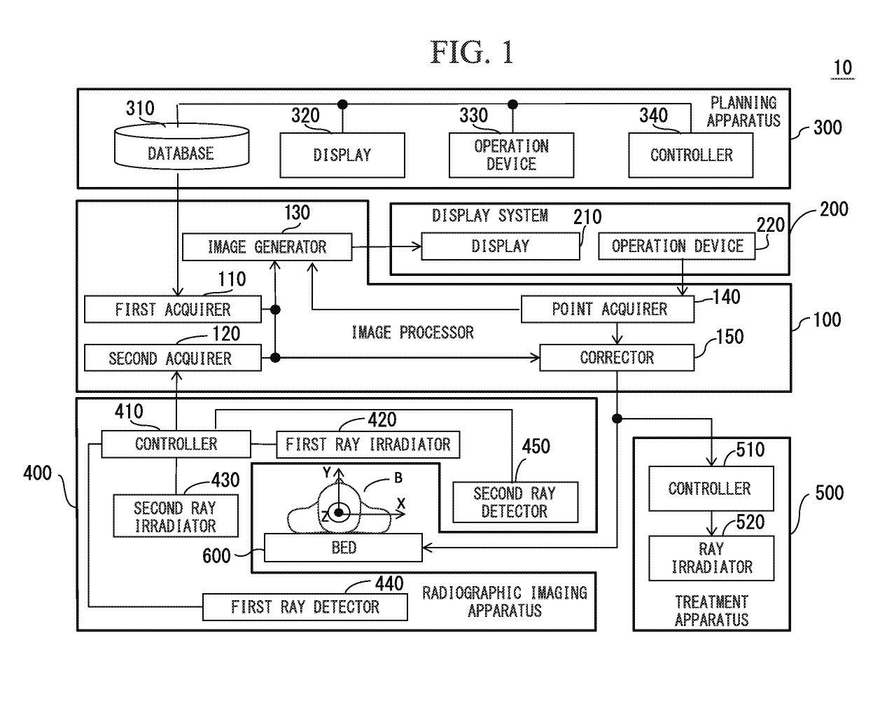

FIG. 1 is a block diagram illustrating an example of a treatment system 10. The treatment system 10 may include, but is not limited to, a radiographic imaging apparatus 400, an image processor 100, and a display system 200. The treatment system 10 may further include, but is not limited to, a planning apparatus 300, a treatment apparatus 500, and a bed 600.

The planning apparatus 300 makes a treatment plan based on an input received through operations by a user or an operator (such as a medical doctor or a surgeon), and images of the inside of a target B to be subject to a radiotherapy, a proton therapy, a particle radiotherapy, or the like. Images are captured using a radiographic imaging apparatus configured to capture a perspective image of the inside of the target B. The radiographic imaging apparatus may be, but is not limited to, an X-ray apparatus, a computed tomography (CT) apparatus, a magnetic resonance imaging (MRI) apparatus, a positron emission tomography (PET) apparatus, a single photon emission computed tomography (SPEC) apparatus, and the like.

The planning apparatus 300 may include, but is not limited to, a database 310, a display 320, an operation device 330, and a controller 340.

The database 310 stores image data of the target B acquired at the time a plan is made. Acquired images may be either two-dimensional or three-dimensional. Image data is data acquired by quantifying, per pixel, a state of the inside of the target B to be treated. The image data may be data based on signals obtained front an X-ray apparatus, a CT apparatus, an MRI apparatus, a PET apparatus, or a SPECT apparatus.

Data stored in the database unit 310 may be voxel data of an acquired, image of the target B. Data stored, in the database unit 310 may also be voxel data as raw data acquired by subjecting projected data to a correction process, such as logarithmic conversion, offset correction, sensitivity correction, beam hardening correction, or scattered radiation correction. Data stored in the database unit 310 may also be two-dimensional image data reconstructed from the voxel data. Descriptions will be given in the present embodiment with respect to a case where the database 310 stores voxel data.

Descriptions will be given in the present embodiment with respect to a case where images acquired by an X-ray CT apparatus at the time a treatment plan is made (hereinafter, “planning time”) are used.

The display 320 displays first and third perspective images under the control of the controller 340 at the planning time. The first perspective image is an image of the target B viewed in a first direction at the planning time. The third perspective image is an image of the target B viewed at the planning time in a third direction different from the first direction. For example, the first and third perspective images are images reconstructed from the voxel data stored in the database 310, that is, digitally reconstructed radiographs (DRR). When there are multiple perspective images each including both negative and positive images, it is preferable to display, under control of the controller 340, images which are inverted so that each image includes either a negative or positive image.

The operation device 330 receives an input through operations by a user. The operation device 330 supplies to the controller 340, a signal in accordance with the received input.

The controller 340 controls each unit included in the planning apparatus 300 based on the signal received from the operation device 330. The controller 340 may be, but is not limited to, a central processing unit (CPU).

The radiographic imaging apparatus 400 captures a perspective image of the inside of the target B at the time of treatment. The radiographic imaging apparatus 400 may be, but is not limited to, an X-ray apparatus, a CT apparatus, an MRI apparatus, and the like. Hereinafter, there will be described a case where the radiographic imaging apparatus 400 is an X-ray imaging apparatus. The radiographic imaging apparatus 400 may include, but is not limited to, a controller 410, and first and second image captures. The first image capture includes a first ray irradiator 420 and a first ray detector 440. The second image capture includes a second ray irradiator 430 and a second ray detector 450.

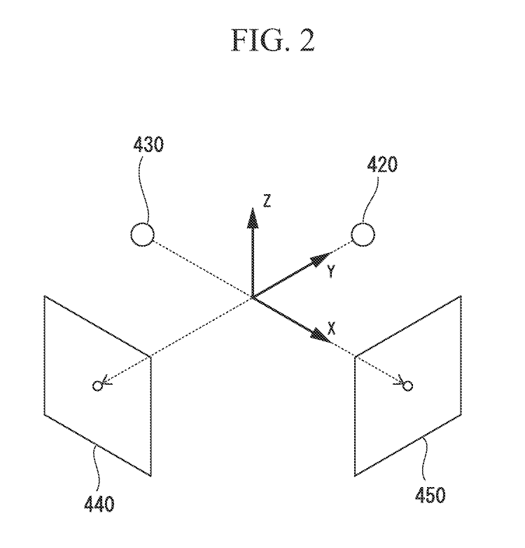

FIG. 2 is a diagram illustrating a positional relationship of the radiographic imaging apparatus 400. The first ray detector 440 may include, but is not limited to, a first flat panel detector (FPD). The first FPD of the first ray detector 440 detects the X-ray projected from the first ray detector 440 and converts the received X-ray into a digital signal. Based on the digital signal converted by the first FPD, the first ray detector 440 generates a second perspective image of the target B. The second perspective image, n a perspective image of the target B viewed at the time of treatment (i.e., at time different from the time the first perspective image is captured) in a second direction that is substantially the same as the first direction in which the first perspective image is viewed. The second perspective image may be, but is not limited to, a ray detected by an radiographic imaging apparatus 400 (X-ray apparatus), that is, an X-ray radiograph (XR). Here, the second, perspective image may be a two-dimensional perspective image reconstructed front voxel data by simulation where positions of X-ray irradiators and projection planes are virtually determined.

The second ray detector 450 may include, but is not limited to, a second FPD. The second FPD of the second ray detector 450 detects an X-ray projected from the second ray detector 450 and converts the received X-ray into a digital signal. Based on the digital signal converted by the second FPD, the second ray detector 450 generates a fourth perspective image of the target B. The fourth perspective image is a perspective image of the target B viewed at the time of treatment (i.e., at time different from the time tire third perspective image is obtained) in a fourth direction that is substantially the same as the third direction in which the third perspective image is viewed.

The first ray detector 440 is disposed so as to be paired with the first ray irradiator 420, thereby enabling the first FPD of the first ray detector 440 to detect an X-ray irradiated from the first ray irradiator 420. The second ray detector 450 is disposed so as to be paired with the second ray irradiator 430, thereby enabling the second FPD of the second ray detector 450 to detect an X-ray irradiated from the second ray irradiator 430.

Configuration and functions of the radiographic imaging apparatus 400 are described here using a three-dimensional coordinate system, such as an XYZ coordinate system shown in FIG 2. The first and second ray detectors 440 and 450 of the radiographic imaging apparatus 400 are subject to calibration in order to obtain a perspective projection matrix used tor coordinate transformation to the XYZ coordinate system. Specifically, the first ray detector 440 is disposed in a negative direction of a Y-axis in the XYZ coordinate system. The second ray detector 450 is disposed in a positive direction of an X-axis in the XYZ coordinate, system. The first ray irradiator 420 is disposed in a positive direction of the Y-axis in the XYZ coordinate system. The first ray irradiator 420 irradiates an X-ray toward the first ray detector 440. The second ray irradiator 430 is disposed in a negative direction of the X-axis in the XYZ coordinate system. The second ray irradiator 430 irradiates an X-ray toward the second ray detector 450.

Although the case where the first and second ray detectors 440 and 450 are disposed so as to be orthogonal to each other has been described in the present embodiment, a configuration of the present embodiment is not limited thereto. To perform a three-dimensional positioning process, however, it is preferable that the first and second ray detectors 440 and 450 are disposed so as not to be parallel to each other.

The X-ray output from the first ray irradiator 420 penetrates the target B and reaches the first ray detector 440. The second perspective image is generated using the energy of the X-ray reaching the first ray detector 440. On the other band, the X-ray output from the-second ray irradiator 430 penetrates the target B and reaches the second ray detector 450. The fourth perspective image is generated using the energy of the X-ray reaching the second ray detector 450.

The controller 410 controls each unit of the radiographic imaging apparatus 400. The controller 410 may be, but is not limited to, a central processing unit (CPU). The controller 410 receives the second perspective image from the first ray detector 440. The controller 410 supplies the second perspective image to the image processor 100.

The image processor 100 generates a first image from the first perspective linage. The image processor 100 generates a second image from the second perspective image. The details of the image processor 100 will be described later.

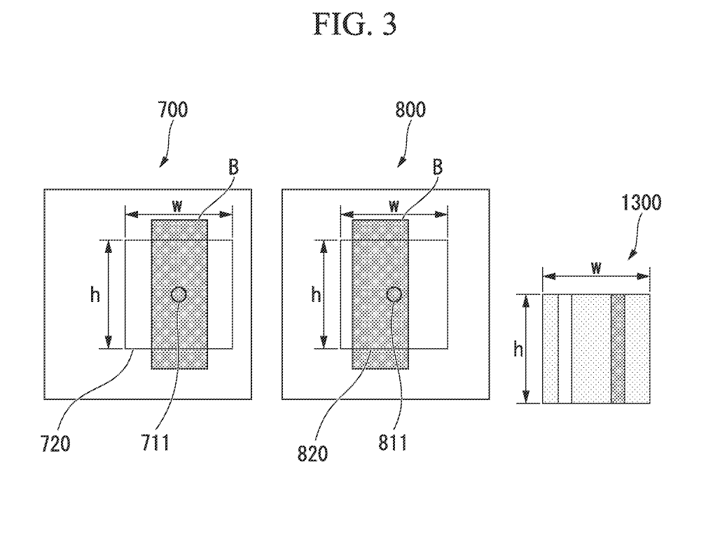

The display system 200 displays, at the time of treatment, images generated by the image processor 100. The display system 200 may include, but is not limited to, a display 210 and an operation device 220. The display 210 receives image data from the image processor 100. The image data may include, but is not limited to, data indicating the first image generated from the first perspective image. The image data may include, but is not limited to, data indicating the second image generated from the second perspective image. The image data may include, but is not limited to, data indicating a similarity image generated from at least a part of the first and second perspective images (which will be described later with reference to FIGS. 3 to 9).

The display 210 displays the first and second images and the similarity image at a predetermined display resolution. The display 210 may be, but is not limited to, a liquid crystal display panel, an organic EL panel, a plasma display panel, or a cathode ray tube (CRT). The predetermined display resolution may be a resolution of the image generated by the image processor 100 or a resolution preciously specified via the operation device 220.

The operation device 220 is an input device that receives an input through operations by the user and supplies the image processor 100 with a signal in accordance with the received input. The operation device 220 may receive an input through operations performed to input to the image processor 100, information indicating a position on the image displayed on the display 210. The information indicating the position on the image may be expressed as a coordinate. The operation device 220 is not limited to a specific device as long as a user can input information indicating a position. The operation device 220 may include, but is not limited to, a touch panel, a keyboard, a mouse, and the like. A user operates, the operation device 220 to designate a position on an image to the precision of the display resolution, thereby inputting information indicating that position to the image processor 100. For example, when the operation device 220 is a mouse, the user moves, using the mouse, a cursor to a position on an image and performs clicking, thereby designating the position on the image.

The operation device 220 and the display 210 may be integrated. In this case, the user touches the operation device 220 displayed on the display 210 to designate a position on an image to the precision of the display resolution, thereby inputting information indicating that position to the image processor 100.

The treatment apparatus 500 is an apparatus to be used for subjecting the target B to a radiotherapy, a proton therapy, or a particle radiotherapy on the target B at the time of treatment. The treatment apparatus 500 may include, but is not limited to, a controller 510 and a ray irradiator 520. The controller 510 controls each unit of the treatment apparatus 500. The controller 510 may be, but is not limited to, a central processing unit. The controller 510 receives a positioning, signal from the image processor 100. Based on the positioning signal the controller 510 controls the ray irradiator 520. The ray irradiator 520, under control of the controller 510, irradiates radio beams, proton beams, or particle beams toward the target B positioned by the bed 600.

The bed 600 receives the positioning signal from the image processor 100. Based on the positioning signal, the bed 600 moves the target B within a predetermined area while keeping, the target B lying. Thus, the radio beams, proton beams, or particle beams are precisely irradiated from the ray irradiator 520 to a point precisely determined in the inside of the target B.

Next, the details of the image processor 100 are described here.

The image processor 100 may include, but is not limited to, a first acquirer 110, a second acquirer 120, an image generator 130, and a point acquirer 140, and a corrector 150.

The first acquirer 110 acquires from the planning apparatus 300, the first perspective image of the target B viewed in the first direction and acquired at a first time. The first acquirer 110 may acquire front the database 310 of the planning apparatus 300, voxel data indicating the first perspective image of the target B. The first acquirer 110 supplies the image generator 130 with image data indicating the first perspective image.

The second acquirer 120 acquires from the controller 410 of the radiographic imaging apparatus 400, the second perspective image of the target B viewed in the second direction and captured at a second time different from the first time. The second direction is substantially the same as the first direction in which the first perspective image is viewed. The second acquirer 120 supplies the second perspective image to the image generator 130 and the corrector 150.

The image generator 130 generates an image with, a predetermined display resolution. The predetermined display resolution may be selected from resolutions available to the display 210 or predetermined by a user.

The image generator 130 receives the first perspective image from the first acquirer 110. The image generator 130 supplies the first perspective image to the corrector 150. Here, the image generator 130 may receive the voxel data from the first acquirer 110. In this case, the image generator 130 reconstructs a first perspective image from the voxel data.

The image generator 130 generates the first image from the first perspective image. The first image is generated by resizing the first perspective image. The term, “resizing” means changing the number of pixels of an image. Here, methods for the image generator 130 to generate the first image include, but are not limited to, nearest neighbor, bi-linear interpolation, cubic convolution, or the like. The image generator 130 supplies the generated first image, to the display 210 of the display system 200.

The image-generator 130 receives the second perspective image from the second acquire 120. The image generator 130 generates the second image from the second perspective image. The second image is generated by resizing the second perspective image. Here, methods for the image generator 130 to generate the second image include, but are not limited to, nearest neighbor, bi-linear interpolation, cubic convolution, or the like. The image generator 130 supplies the generated second image to the display 210 of the display system 200.

The image generator 130 generates, as an image based on the first and second perspective images, a image based on a similarity between the first and second perspective images (hereinafter, “similarity image”). The similarity image may be an image based on a similarity between the first and second images respectively generated from the first and second perspective images. The image generator 130 generates the similarity image, based on the first and second perspective images, wherein a first coordinate of the first perspective image is unchanged, and a second coordinate of the second perspective image is changed. The first perspective image is fixed on the first coordinate. The second perspective image is fixed on the second coordinate. The second coordinate is changeable or movable by using the operation device 220. For example, by using the operation device 220, the second coordinate is moved to reduce a difference between the first and second positions of the first and second points on the first aid second coordinates, respectively, thereby increasing the similarity. The second coordinate can be further moved so that the first and second positions on the first and second coordinates are identical to each other, to take the maximum similarity.

The image generator 130 generates, as the similarity image, a differential image between the first and second perspective images. The image generator 130 renders the display 210 display the first and second perspective images and the differential image in a line. The image generator 130 may have the display 210 display a differential image superimposed on the first or second perspective image (hereinafter, “superimposed image”).

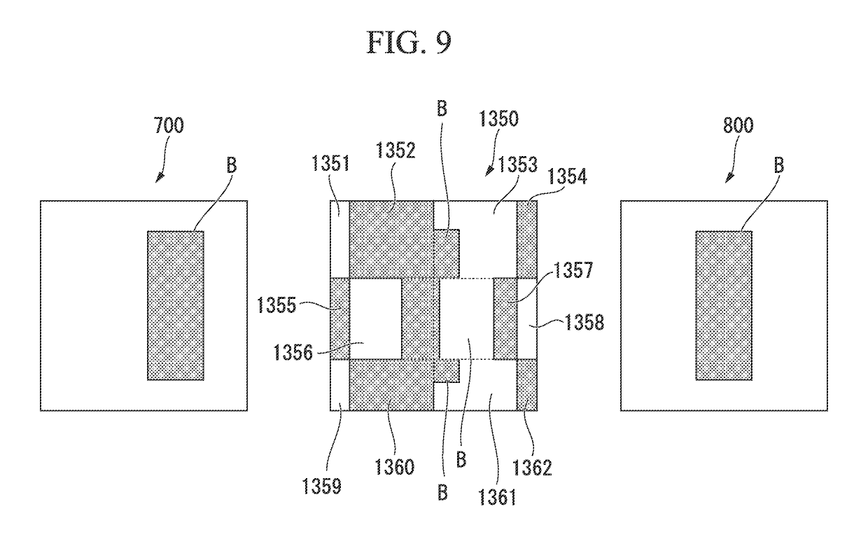

The image generator 130 may generate, as the similarity image, a patched image or a switching image based on the first and second perspective images. The patched image is art image where parts of the first and second perspective images are arranged in a grid. The patched image will be described later with reference to FIG. 9. The switching image is an image where the first and second perspective images alternately switches with a predetermined period. The switching image will be described later with use of Expression (3).

Additionally, the image generator 130 may generate, as the similarity image, an image obtained from a square sum of a difference in pixel value between the first and second perspective images, which is referred to as a sow of squared difference (SSD) map. Further, the image generator 130 may generate, as the similarity image, an image obtained from an absolute sum of the difference in pixel value between the first and second perspective images, which is referred to as a sum of absolute difference (SAD) map. Moreover, the image generator 130 may generate, as the similarity image, an image obtained from normalized mutual correlation between pixel values of the first and second perspective images, which is referred to as a normalized mutual correlation map. Additionally, the linage generator 130 may generate, as the similarity image, an image based on the first and second perspective images which are rotated, increased or decreased in size. Here, the image generator 130 may generate, as the similarity image, an image based on the first and second images which are rotated, increased or decreased in size, in lieu of the first and second perspective images which are rotated, increased or decreased in size.

The image generator 130 receives from the point acquirer 140, information indicating a position of a first point. The image generator 130 may generate an image obtained by superimposing the image representing the position of the first, point on the first image. The image representing the position is denoted by a while circle or the like. The image generator 130 receives from the point acquirer 140, information indicating a position of a second point. The image generator 130 may generate an image obtained by superimposing the image representing the position of the second point on the second image.

The image generator 130 may generate a frame image representing an outline of an image based on the first and second perspective images. The image based on the first and second perspective images may be, but is not limited to, the similarity image. Here, the image generator 130 may change a color of the frame image in accordance with the similarity between the first and second perspective images, that is, the similarity between the first and second images.

The point acquirer 140 acquires from fire operation device 220, the first point designated on the first image. The point acquirer 140 may acquire information indicating an updated position of the first point. The point acquirer 140 supplies first information indicating the position of the first point to the corrector 150 and the image generator 130.

The point acquirer 140 acquires from the operation device 220, a second point (corresponding point) on the second image, which corresponds to the first point (designated point) on the first image. The point acquirer 140 may acquire information indicating an updated position of the second point. The point acquirer 140 supplies second information indicating the position of the second point to the corrector 150 and the image generator 130.

The corrector 150 receives the first information indicating the position of the first point and the second information indicating the position of the second point. Based on the first and second information, the corrector 150 generates a positioning signal and supplies the generated positioning signal to the treatment apparatus 500 and the bed 600.

The corrector 150 receives the first perspective image from the image generator 130. The corrector 150 receives the second perspective image from the second acquirer 120. The corrector 150 determines a similarity between a partial image of the first perspective image and a partial image of the second perspective image. The similarity is calculated using image processing, such as pattern matching. A positional relationship between a point on the first perspective image and a point on the second perspective image can be obtained precisely.

Next, the details of the image generator 130 are described here.

Hereinafter, “I₁(x, y)” represents a pixel value of a coordinate (x, y) on the first image. “I₂(x, y)” represents a pixel value of a coordinate (x, y) on the second image. “(x₁, y₁)” represents a coordinate of the first point on the first image. “(x₂, y₂)” represents a coordinate of the second point on the second image. “A(x, y)” represents a pixel value of a coordinate (x, y) on the similarity image. “h” represents the vertical size of the similarity image. “w” represents the horizontal sixe of the similarity image.

### (Differential Image)

FIG. 3 is a diagram illustrating an example of an image including the first and second images and the differential image aside differential image with lower similarity, wherein those images are displayed without overlay or superimposition with each other. A first region 720 is a region of the first image 700 and has a center at a first point 711. The size of the first region 720 is h(vertical)×w(horizontal). A similarity image 1300 shown in FIG. 3 has a low similarity between the perspective image of the target B included in the first region 720 and the perspective image of the target B included in the second region 820. When the similarity is low, there is a large difference in position between the first point 711 (designated point) and the second point 811 (corresponding point) which are included in the inside of the target B.

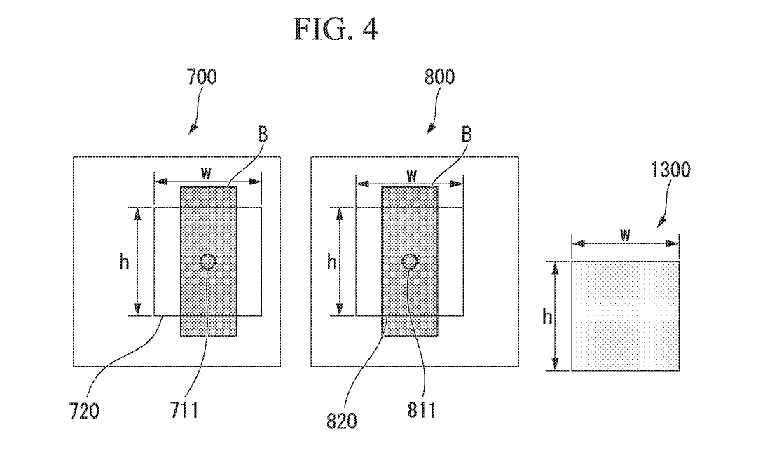

FIG. 4 is a diagram illustrating an example of an image including the first and second images and the differential image as the differential image with higher similarity, which are arranged in a line. A second region 820 is a region of the second image 800 and has a center at a second point 811. The size of the second region 820 is h(vertical)×w(horizontal). It is assumed here that a position of the second point 811 on the second image 800 shown in FIG. 4 is different from that shown in FIG. 3. A similarity image 1300 shown in FIG. 4 has a high similarity between the perspective image of the target B included in the first region 720 and the perspective image of the target B included in the second region 820. The designated point and the corresponding point anatomically represent the same portion of the inside of the target B. For this reason, when the similarity is high, it is estimated that there is a small difference in position between the first point 711 (designated point) and the second point 811 (corresponding point) which are included in the inside of the target B.

The image generator 130 renders the display unit 210 display at the predetermined display resolution, the generated first image 800, the second image 800, and the similarity image 1300, which are arranged in a line. The first image 700 is an image generated from the first perspective image. The second image 800 is an image generated from the second perspective image. The perspective image of the target B viewed in the first direction is displayed on the first image 700. The perspective image of the target B viewed in the second direction that is substantially the same as the first direction is displayed on the second image 800. For example, the first and second directions are the negative direction of the Y axis shown in FIG. 2.

The position (x₁, y₁) of the first point 711 (designated point) on the first image 700 is designated by the user operating the operation device 220. The operation device 220 supplies the image processor 100 with first information indicating the position of the first point 711. Additionally, the position (x₂, y₂) of the second point 811 (corresponding point) on the second image 800, which is anatomically the same position as that, of the first point 711, is designated, by the user operating the operation device 220. The operation device 220 supplies the image processor 100 with second information indicating the position of the second point 811.

The position, of the first point 711 may be updated on the first image 700. Similarly, the position of the second point 811 maybe updated on the second image 800. When the operation device 220 is a mouse, for example, the user moves, using the mouse, a cursor to the position of the first point 711 on the first image 700 or the second point 811 on the second image 800, and drags the first point 711 or the second point 811, thus updating the position of the first point 711 or the second point 811. When the operation device 220 is a keyboard, for example, the user presses an arrow key on the keyboard to select the first point 711 or the second point 811, thereby updating the position of the first point 711 or the second point 811.

Here, the first information indicating the position of the first point 711 and the second information indicating the position of the second point 811 may be received horn another database and be supplied to the point acquirer 140 with higher precision than that of the display resolution. For example, the first and second information may be supplied to the point acquirer 140 not through operations by the user, but from the other database. For example, the point acquirer 140 may acquire the first and second information horn a database (data server), a recording medium such as a CD (compact disc) or a DVD (digital versatile disc), or a network storage. Further, the point acquirer 140 may acquire the first and second information respectively indicating the positions of the first and second points 711 and 811 detected by image processing such as pattern matching.

The similarity image 1300 is a differential image based on the similarity between the first and second perspective images. In lieu of the differential image based on the similarity between the first and second perspective images, the similarity image 1300 may be a differential image based on the similarity between the first image 700 and the second image 800 respectively generated from the first and second perspective images. The similarity image 1300 is expressed by Expression (1).

$$A ( x , y ) = {I}_{1} ( {x}_{1} - \frac{w}{2} - x , {y}_{1} - \frac{h}{2} - y ) - {I}_{2} ( {x}_{2} - \frac{w}{2} - x , {y}_{2} - \frac{h}{2} - y ) \qquad ( 1 )$$

As the difference in position between the first point 711 and the second point 811 which are included in the inside of the target B is smaller, there is a higher similarity between an image close to the first point 711 and an image close to the second point 811, that is, between an image in the first region 720 and an image in the second region 820. In FIG. 4, there is a high similarity between the image in the first region 720 and the image in the second region 820, thereby making the similarity image 1300 flat.

Accordingly, the user can update, by operating the operation device 220, the position of the first point 711 on the first image 700 or the second point 811 on the second image 800 so that the similarity image 1300 becomes flat. When the operation device 220 is a mouse, for example, the user moves, using the mouse, a cursor to the position of the first point 711 on the first image 700 or the second point 811 on the second image 800, and drags the first point 711 or the second point 811, thus updating the position of the first point 711 or the second point 811. Thus, the precision of the process by the user of inputting the position of the first point 711 or the second point 811 is enhanced.

To determine the position of a patient, a user (such as a surgeon) manually designates, based on visual determination by the user, corresponding points on an X-ray radiographic image and an X-ray referential image. In other words, the user manually selects corresponding points. For this reason, the designated corresponding points on tire X-ray radiographic image and the X-ray referential image do not always indicate the anatomically same position. Thus, there is a difference in position between the corresponding ponds designated on the X-ray radiographic image and the X-ray referential image.

Additionally, as explained above, the image generator 130 may have the display 210 display a frame image representing the outline of the similarity image 1300. In this case, a color of the frame image may be changed in accordance with the flatness of the similarity image 1300, that is, the similarity between an image in the first region 720 and an image in the second region 820. Thus, the precision of the process by the user of inputting the position of the first point 711 or the second point 811 is enhanced.

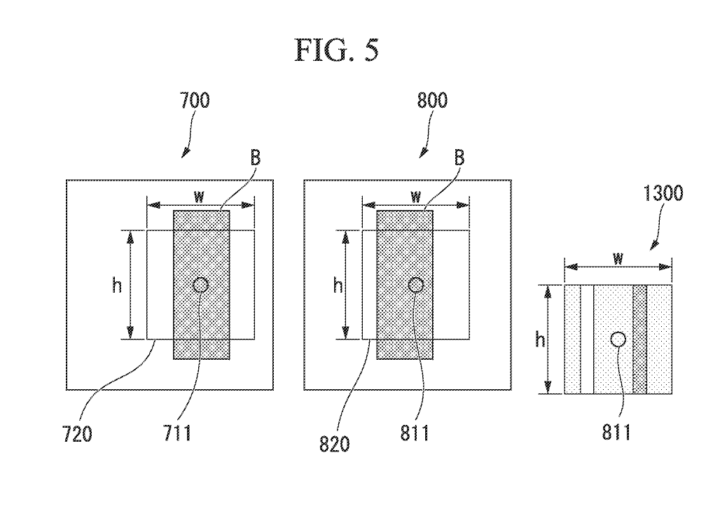

FIG. 5 is a diagram illustrating a state of the differential image as the similarity image on which an image representing the position of a point is superimposed. The image generator 130 may superimpose on the second image 800 and the similarity image 1300, an image representing a position of the second point 811 (represented by a white circle or the like). Additionally, the display 210 may superimpose on the second image 700 and the similarity image 1300, an image representing a position of the first point 711.

The vertical and horizontal size h×w (magnification ratio) of the similarity image 1300 may be changeable. When the similarity image 1300 is displayed in reduced size, it is easy for the user to move by a large amount, by operating the operation device 220, the position of the first point 711 or the second point 811 on the similarity image 1300. On the other hand, when the similarity image 1300 is displayed in increased size, it is easy for the user to move with high resolution, by operating the operation device 220, the position of the first point 711 or the second point 811 on the similarity linage 1300. The display 210 may display information (x₁, y₁) representing the position of the first point 711 and information (x₂, y₂) representing the position of the second point 811.

### (Superimposed Image)

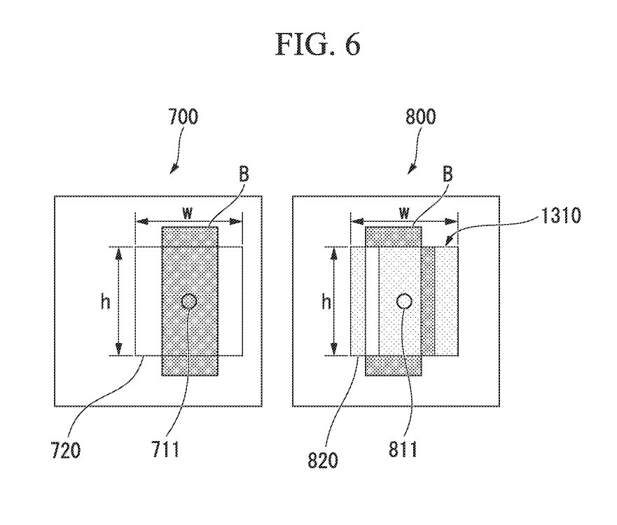

FIG 6 is a diagram illustrating a displayed image in which a superimposed image as a similarity image is superimposed on the second image 800. The image generator 130 renders the display 210 display, at a predetermined display resolution, the generated first and second images 700 and 800 which are arranged in a line. Preferably, the image generator 130 renders the display 210 superimpose the similarity image 1310 on the first image 700 or the second image 800. Thus, the second region 820 to be compared to the first region 720 is replaced with the similarity image 1310, thereby making it possible for the user to intuitively recognize the difference in position between the designated corresponding points. In the case of FIG. 6, the image generator 130 renders the display 210 display the first image 700, the second image 800, and the similarity image 1310 superimposed on the second region 820. The similarity image 1310 can be expressed by Expression (2).

$$A ( x , y ) = α {I}_{1} ( {x}_{1} - \frac{w}{2} - x , {y}_{1} - \frac{h}{2} - y ) + ( 1 - α ) {I}_{2} ( {x}_{2} - \frac{w}{2} - x , {y}_{2} - \frac{h}{2} - y ) \qquad ( 2 )$$

Here, α denotes a ratio at the time the image is superimposed, which is a value such that “0≦α1”.

(Similarity image Based on Image Rotated, Increased or Decreased in Size)

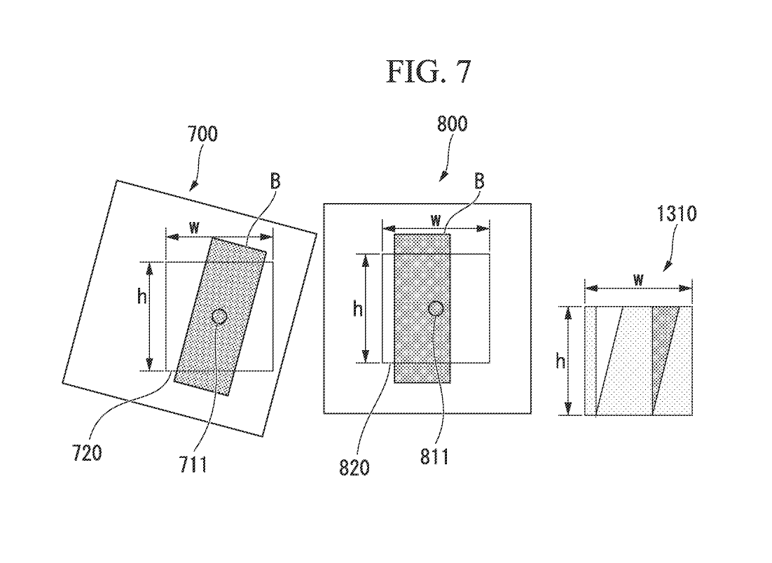

FIG. 7 is a diagram illustrating a displayed image including the first image, the second image, and a similarity image based on an image rotated, increased or decreased in size, which are arranged in a line. When a difference in position of the target B between on the first image 700 and on the second image 800 is larger than a predetermined threshold, the image generator 130 may generate the similarity image 1310 based on the first image 700 or the second image 800 which is rotated, increased or decreased in size.

Here, the image generator 130 may rotate, increase or decrease in size, the first image 700 or the second image 800, based on a conversion amount determined by the user. Additionally, the image generator 130 may select from among multiple predetermined conversion amounts, a conversion amount that reduces the difference in position the most. In this case, the difference in position may be evaluated based on an BSD between an image on the first region 720 and an image in the second region 820. In that case, as the SSD is smaller, the difference in position is estimated to be smaller.

### (Switching Image)

The first image 700, the second image 800, or the similarity image 1310 may be expressed by Expression (3).

$$A ( x , y ) = \{ \begin{matrix}{I}_{1} ( {x}_{1} - \frac{w}{2} - x , {y}_{1} - \frac{h}{2} - y ) & ( F = 0 ) \\ {I}_{2} ( {x}_{2} - \frac{w}{2} - x , {y}_{2} - \frac{h}{2} - y ) & ( F = 1 )\end{matrix} \qquad ( 3 )$$

Here, F represents a value of a flag (0 or 1). The image generator 130 may switch the value of the flag with a predetermined period of time. Thus, the image generator 130 can switch, as time passes, an image to be displayed, from among the first image 700, the second image 800, and the similarity image 1310. Additionally, the image generator 130 may switch the value of the flag at the timing dial is based on an operation by the user.

### (SSD Map)

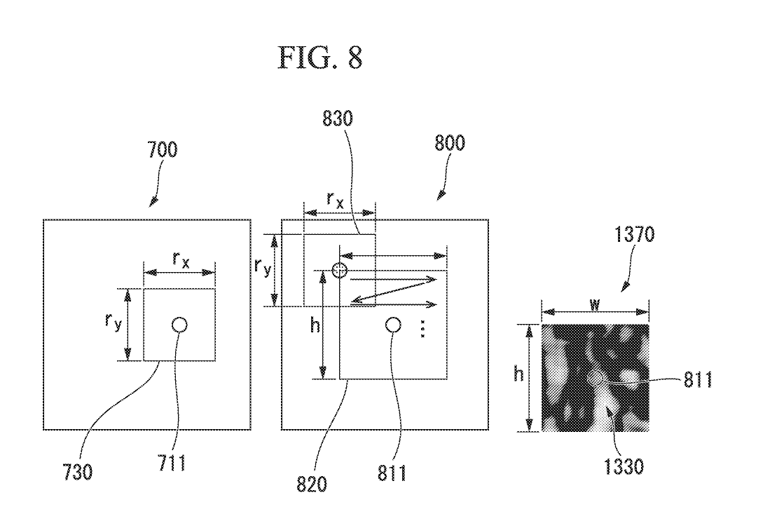

FIG. 8 is a diagram illustrating a displayed image including the first image, the second image, and an SSD map as the similarity image, which are arranged in a line. The image generator 130 renders the display 210 display at a predetermined display resolution, the generated first image 700, the second image 800, and a similarity image 1370, which are arranged in a line. A third region 730 of the first image 700 is defined. The third region 730 has a center at the first point 711 and the horizontal and vertical size rₓ×r_{y}. On the other hand, a fourth region 830 of the second image 800 is defined as a region to be used to calculate an SSD. The fourth region 830 has the horizontal and vertical size rₓ×r_{y}.

The image generator 130 raster-scans the fourth region 830 with respect to the second region 820 to calculate an SSD between the third region 730 and the fourth region 830, thus generating the similarity image 1370 based on the calculated SSD. The similarity image 1370 can be expressed by Expression (4).

$$A ( x , y ) = {\sum }_{{r}_{x} , {r}_{y} ∈ R} {( {I}_{1} ( {x}_{1} - {r}_{x} , {y}_{1} - {r}_{y} ) - {I}_{2} ( {x}_{2} - \frac{w}{2} - {r}_{x} , {y}_{2} - \frac{h}{2} - y - {r}_{y} ) )}^{2} \qquad ( 4 )$$

On the similarity image 1370, a pixel at the position which causes a smaller SSD is displayed in brighter color (such as white). Additionally, on the similarity image 1370, a pixel at the position which causes a larger SSD is displayed in darker color (such as black). An SSD minimum position 1330 is the position of a pixel on the similarity image 1370, which causes the smallest SSD. In FIG. 8, the pixel at the SSD minimum position 1330 is displayed in the brightest color (white) on the similarity image 1370. Here, a relationship between the SSD and the brightness is not limited to the above, and a relationship therebetween may be reverse to the above relationship. Additionally, the relationship between the SSD and the brightness may be configured such that pixels of a color image are displayed in different colors in accordance with the SSD.

Based on information concerning the SSD shown on the similarity image 1370, the user can align the second point 811 to the SSD minimum position 1330 by operating the operation device 220. When the operation device 220 is a mouse, for example, the user moves, using the mouse, a cursor to the image of the second point 811 on the second image 800, and drags the second point 811, thus updating the position of the second point 811. Thus, the precision of the process by the user of inputting the position of the second point 811 representing the position of the inside of the target B is enhanced.

The vertical and horizontal size h×w (magnification ratio) of the similarity image 1370 may be changeable. Additionally, the image generator 130 may render the display 210 superimpose the similarity image 1370 on the second region 820. Here, the linage generator 130 may have the display 210 display the similarity image 1370 obtained by adjusting pixel values in accordance with the ratio a that is a value such that “0≦α≦1”. This alternative configuration is applicable to an SAD map and a normalized mutual correlation map, which will be described below. Thus, the user can simultaneously view, on the display 210, both the second image 800 (original image) and the superimposed similarity image 1370.

### (SAD Map)

The similarity image 1370 may be expressed by Expression (5).

$$A ( x , y ) = {\sum }_{{r}_{x} , {r}_{y} ∈ R} \vert {I}_{1} ( {x}_{1} - {r}_{x} , {y}_{1} - {r}_{y} ) - {I}_{2} ( {x}_{2} - \frac{w}{2} - {r}_{x} , {y}_{2} - \frac{h}{2} - y - {r}_{y} ) \vert \qquad ( 5 )$$

The image generator 130 renders the display 210 display, at a predetermined display resolution, the generated first image 700, the second image 800, and the similarity image 1370, which are arranged in a line. The third region 730 of the first image 700 is defined as a region to be used to calculate an SAD. The third region 730 has a center at the first point 711 and the horizontal and vertical size rₓ×r_{y}. On the other hand, the fourth region 830 of the second image 800 is defined, as a region to be used to calculate an SAD. The fourth region 830 has the horizontal and vertical size rₓ×r_{y}.

### (Normalized Mutual Correlation Map)

The similarity image 1370 may be expressed by Expression (6).

$$A ( x , y ) = \frac{\begin{matrix}{\sum }_{{r}_{x} , {r}_{y} ∈ R} {I}_{1} ( {x}_{1} - {r}_{x} , {y}_{1} - {r}_{x} ) \\ {I}_{2} ( {x}_{2} - \frac{w}{2} - x - {r}_{x} , {y}_{2} - \frac{h}{2} - y - {r}_{y} )\end{matrix}}{\sqrt{\begin{matrix}{\sum }_{{r}_{x} , {r}_{y} ∈ R} {{I}_{1} ( {x}_{1} - {r}_{x} , {y}_{1} - {r}_{y} )}^{2} \\ {\sum }_{{r}_{x} , {r}_{y} ∈ R} {{I}_{2} ( {x}_{2} - \frac{w}{2} - x - {r}_{x} , {y}_{2} - \frac{h}{2} - y - {r}_{y} )}^{2}\end{matrix}}} \qquad ( 6 )$$

The image generator 130 renders the display 210 display, at a predetermined display resolution, the generated first image 700, the second image 800, and the similarity image 1370, which are arranged in a line. The third region 730 of the first image 700 is defined as a region to be used to calculate a normalized mutual correlation between pixel values. The third region 730 has a center at the first point 711 and the horizontal and vertical size rₓ>r_{y}. On the other hand, the fourth region 830 of the second image 800 is defined as a region to be used to calculate a normalized mutual correlation between pixel values. The fourth region 830 has the horizontal and vertical size rₓ×r_{y}.

### (Patched Image)

FIG. 9 is a diagram illustrating a displayed image including the first image, the second image, and a patched image as a similarity image, which are arranged in a line. A similarity image 1350 is an image obtained by arranging in a grid, image regions extracted from the second image 800 and image regions extracted from the first image 700 having been subjected to negative-positive inversion. In the case of FIG. 9, the similarity image 1350 includes image regions 1351, 1353, 1356, 1358, 1359, and 1361 which are extracted from the second image 800, and image regions 1352, 1354, 1355, 1357, 1359, 1360, and 1362 which are extracted from the first image 700 having been subjected to negative-positive inversion. When it is assumed that both the first image 700 and the second image 800 are flat images in white, the similarity image 1350 may be configured such that multiple image regions are displayed in a checkerboard pattern using white and black. The similarity image 1350 is expressed by Expression (7).

$$A ( x , y ) = \{ \begin{matrix}{I}_{1} ( {x}_{1} - \frac{w}{2} - x , {y}_{1} - \frac{h}{2} - y ) & ( M ( x , y ) = 0 ) \\ {I}_{2} ( {x}_{2} - \frac{w}{2} - x , {y}_{2} - \frac{h}{2} - y ) & ( M ( x , y ) = 1 )\end{matrix} \qquad ( 7 )$$

Here, M(x, y) represents a mask label that has a value 0 or 1. When the operation device 220 is a mouse, for example, the user moves, using the mouse, a cursor to each image region of the similarity image 1350 on the similarity image 1350, and drags the image region, thus moving a boundary between adjacent image regions of the similarity image 1350. Additionally, only the similarity image 1350 may be displayed on the display 210, instead of being displayed with the first image 700 and the second image 800.

Based on the boundaries among adjacent image regions of the similarity image 1350, the user can easily recognize a difference in position of the target B between on the first image 700 and on the second image 800. Thus, the precision of a process by the user of inputting a position of a point representing a position of the inside of the target B is enhanced.

Here, the configurations described above with respect to the differential image, the superimposed image, the similarity image based on the rotated image or the like, the switching image, the SSD map, the SAD map, the normalized mutual correlation map, and the patched image, may be combined. For example, the similarity image based on the rotated image or the like may be an SAD map.

Next, procedure for the processing of the image processor 100 is described here.

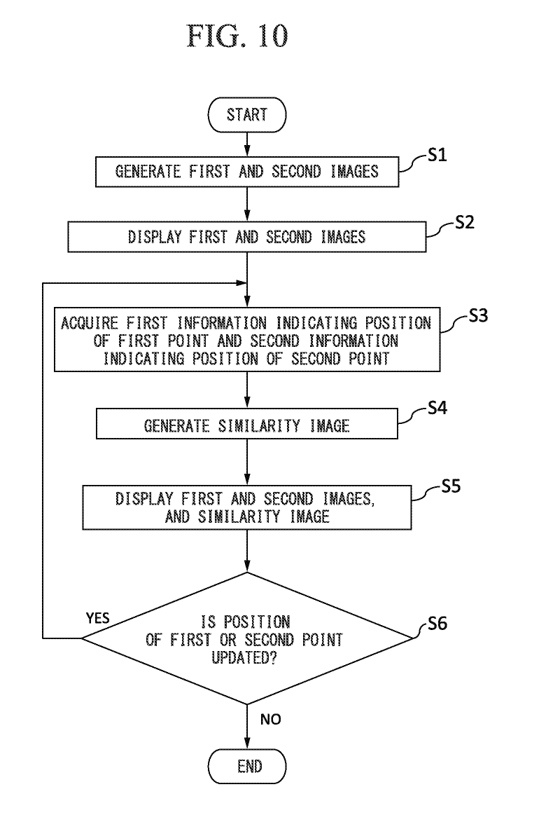

FIG. 10 is a flowchart illustrating an example of procedure for the processing of the imago processor 100.

(Step S1) The first acquirer 110 acquires from the planning apparatus 300, the first perspective image of the target B viewed in the first direction and acquired at the first time. The first perspective image may be voxel data of the target B. The second acquirer 120 acquires from the radiographic imaging apparatus 400, the second perspective image of the target B viewed in the second direction that is substantially the same as the first direction and acquired at the second time different from the first time. The image generator 130 generates the first image 700 from the first perspective image. The image-generator 130 generates the second linage 800 from the second perspective image.

(Step S2) The image generator 130 renders the display 210 display the first image 700 and the second image 800.

(Step S3) The point acquirer 140 acquires the first information indicating the position of the first point 711 designated on the first image 700. The point acquirer 140 acquires the second information indicating the position of the second point 811 designated on the second image 800, which corresponds to the first point 711 on the first image 700.

(Step S4) The image generator 130 generates the similarity image (such as the similarity image 1300, 1310, 1350, or 1370, or the switching image).

(Step S5) The image generator 130 renders the display 210 display the generated first image 700, the second image 800, and the similarity image at the predetermined display resolution.

(Step S6) The point acquirer 140 determines whether or not the first information indicating the position of the first point 711 or the second information indicating the position of the second point 811 has been updated, if it is determined that the first or second information has been updated (step S6: YES), the processing returns to step S3. On the other hand, if it is determined that none of the first and second information has been updated (step S6: NO), the processing ends.

According to the image processor of at least one embodiment described above, it is possible to generate an image to be displayed for a user to easily recognize a relationship among corresponding characteristic points designated on multiple respective images.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

For example, the position of the first point 711 (corresponding point) may be designated on the second image 800, instead of being designated on the first image 700. In this case, the second point 811 (designated point) is designated on the first image 700, instead of being designated on the second image 800.

What is claimed is:

1. An image processor comprising:
a first acquirer that acquires a first perspective image of a target viewed in a first direction;
a second acquirer that acquires a second perspective image of the target viewed in a second direction at a time different from when the first acquirer acquires the first perspective image, the second direction being substantially the same as the first direction;
a point acquirer that acquires a first information indicating a first position of a first point on the first perspective image, and a second information indicating a second position of a second point on the second perspective image; and
an image generator that generates a first image that is based on the first and second perspective images, wherein a first coordinate of the first perspective image is changed so that the first point corresponds to the second point.

2. The processor according to claim 1, wherein the point acquirer acquires at least one of an updated one of the first information indicating the first position updated, and an updated one of the second information indicating the second position updated, and
the image generator updates the first image based, on the updated ones of the first and second informations.

3. The processor according to claim 1, wherein the image generator generates a second image that is based on an image In a first region of the first perspective image and based on an image in a second region of the second perspective image,
the image in the first region includes the first point, and the image in the second region includes the second point.

4. The processor according to claim 2, wherein the image generator generates a second image that is based on an image in a first region of the first perspective image and based on an image in a second region of the second perspective image,
the image in the first region includes the first point, and the image in the second region includes the second point,

5. The processor according to claim 3, wherein the image generator superimposes, on the second image, any one of a first mark specifying the first position and a second mark specifying the second position.

6. The processor according to claim 4, wherein the image generator superimposes, on the second image, any one of a first mark specifying the first position and a second mark specifying the second position.

7. The image processor according to claim 1, wherein the image generator generates any one of: a differential image between the first and second perspective images; a superimposed image of the differential image over the first image; and an image having a plurality of sectioned regions, each of which is a corresponding part of the first or second perspective image.

8. The image processor according to claim 2, wherein the image generator generates any one of a differential image between the first and second perspective images; a superimposed image of the differential image over the first image; and an image having a plurality of sectioned regions, each of which is a corresponding part of the first or second perspective image.

9. The image processor according to claim 3, wherein the image generator generates any one of: a differential image between the first and second perspective images; a superimposed image of the differential image over the first image; and an image having a plurality of sectioned regions, each of which is a corresponding part of the first or second perspective image.

10. The image processor according to claim 1, wherein the image generator generates any one of an image generated using a square sum of a difference in pixel value between the first and second perspective images; an image generated using an absolute sum of the difference in pixel value; and an image generated using a normalized mutual correlation between pixel values of the first and second perspective images,

11. The image processor according to claim 2, wherein the image generator generates any one of: an image generated using a square sum of a difference in pixel value between the first and second perspective images; an image generated using an absolute sum of the difference in pixel value; and an image generated using a normalized mutual correlation between pixel values of the first and second perspective images.

12. The image processor according to claim 3, wherein the image generator generates any one of an image generated using a square sum of a difference in pixel value between the first and second perspective images; an image generated using an absolute sum of the difference in pixel value; and an image generated using a normalized mutual correlation between pixel values of the first and second perspective images.

13. The image processor according to claim 1, wherein the image generator generates at image based on the first or second perspective image that is rotated or changed in size.

14. The image processor according to claim 2, wherein the linage generator generates an image based on the first or second perspective image that is rotated or changed in size.

15. The image processor according to claim 3, wherein the linage generator generates an image based on the first or second perspective image that is rotated or changed in size.

16. The image processor according to claim 4, wherein the image generator generates an image based on the first or second perspective image that is rotated or changed in size.

17. The image processor according to claim 1, wherein the image generator generates a frame image representing the outline of the first image.

18. The image processor according to claim 17, wherein the image generator changes a color of the frame image in accordance with a similarity between the first and second perspective images.

19. A treatment system comprising:
an image processor;
a radiographic imaging apparatus;
a display system;
an operation device; and
a treatment apparatus,
wherein the image processor comprises:
a first acquirer that acquires a first perspective image of a target viewed in a first direction;
a second acquirer that acquires a second perspective image of the target viewed in a second direction at a time different from when the first acquirer acquires the first perspective image, the second direction being substantially the same as the first direction;
a point acquirer that acquires first information indicating a first position of a first point on the first perspective image, and a second information indicating a second position of a second point on the second perspective image; and
an image generator that generates a first image that is based on the first and second perspective images, wherein a first coordinate of the first perspective image is changed so that the first, point corresponds to the second point,
wherein, the radiographic imaging apparatus, captures the first or second perspective image of the target,
wherein the display device displays the first and second perspective images,
wherein the operation device is used to input the first and second information to the point acquirer, and
wherein the treatment apparatus is used to perform a treatment on the target based on the first image.

20. An image processing method comprising:
acquiring a first perspective image of a target viewed in a first direction;
securing a second perspective image of the target viewed in a second direction at a time different from when the first acquirer acquires the first perspective image, the second direction being substantially the same as the first direction;
acquiring first information indicating a first position of a first point on the first perspective image, and a second information indicating a second position of a second point on the second perspective image; and
generating a first image that is based on the first and second perspective images, wherein a first coordinate of the first perspective image is changed so that the first point corresponds to the second point.
